(19) United States
(12) Patent Application Publication
(10) Pub. No.: US 2007/0043444 A1
(43) Pub. Date: Feb. 22, 2007

(54) Prosthetic device

(76) Inventor: Don Kevin Lester, Fresno, CA (US)

Correspondence Address: FISH & RICHARDSON P.C., PO BOX 1022, MINNEAPOLIS, MN, 55440-1022, US

(21) Appl. No.: 11/210,067

(22) Filed: Aug. 22, 2005

(51) Int. Cl. A61F 2/38; A61F 2/30

(52) U.S. Cl. 623/20.15; 623/23.39

(57) ABSTRACT

A prosthetic device and method for implanting the prosthetic device into a patient are described. The prosthetic device includes a first prosthetic component configured to be implanted into a patient while disconnected from a second prosthetic component, and a second prosthetic component configured to be implanted into a patient while disconnected from the first prosthetic component. The first and second components forming the prosthetic device are connected to one another in situ within the patient.

### TECHNICAL FIELD

This invention relates to a medical device and process for implanting a medical device.

### BACKGROUND

Prosthetic devices are routinely implanted into patients, including various joint replacements, such as knee and hip replacements. An appropriately sized and shaped prosthetic device is selected to accommodate the particularities of the patient. An incision is made on the patient, and the prosthetic device is surgically implanted into the patient. For example, a conventional knee replacement includes a femur portion that is attached to the patient's femur and a tibia portion that is attached to the patient's tibia. The femur and tibia portions mate to artificially perform the functions of the patient's knee.

### SUMMARY

This invention relates to a medical device and process for implanting the medical device. In general, in one aspect, the invention features a prosthetic device including a first prosthetic component configured to be implanted into a patient while disconnected from a second prosthetic component, a second prosthetic component configured to be implanted into a patient while disconnected from the first prosthetic component, and one or more connectors configured to connect the first prosthetic component to the second prosthetic component after the first and the second prosthetic components are positioned within the patient. The prosthetic device is sagittally split into the first and the second prosthetic components.

Implementations of the invention can feature one or more of the following. The prosthetic device can be a knee replacement device including a femur portion. The femur portion can include the first prosthetic component connected to the second prosthetic component and be configured to connect to the patient's femur.

The prosthetic device can further include a tibia portion configured to connect to the patient's tibia. The tibia portion can include a base where the base includes a third prosthetic component configured to be implanted into the patient while disconnected from a fourth prosthetic component, a fourth prosthetic component configured to be implanted into the patient while disconnected from the third prosthetic component, and one or more second connectors configured to connect the third prosthetic component to the fourth prosthetic component after the third and the fourth prosthetic components are positioned within the patient. The base is sagittally split into the third and the fourth prosthetic components. The tibia portion can further include a cap where the cap includes a fifth prosthetic component configured to be implanted into the patient, and configured to connect to the third and fourth prosthetic components while within the patient. The one or more connectors and/or second connectors can be screws.

In general, in another aspect, the invention features a knee replacement device including a first prosthetic component configured to be implanted into a patient while disconnected from a second prosthetic component, a second prosthetic component configured to be implanted into a patient while disconnected from the first prosthetic component and one or more first connectors configured to connect the first prosthetic component to the second prosthetic component after the first and the second prosthetic components are positioned within the patient. The knee replacement device includes a femur portion and the femur portion is sagittally split into the first prosthetic component and the second prosthetic component. The knee replacement device further includes a third prosthetic component configured to be implanted into a patient while disconnected from a fourth prosthetic component, a fourth prosthetic component configured to be implanted into a patient while disconnected from the third prosthetic component, and one or more second connectors configured to connect the third prosthetic component to the fourth prosthetic component after the third and the fourth prosthetic components are positioned within the patient. The knee replacement device further includes a tibia portion and the tibia portion is sagittally split into the third prosthetic component and the fourth prosthetic component.

Implementations of the invention can include one or more of the following. The knee replacement device can further include a fifth prosthetic component configured to be implanted into the patient, and configured to connect to the third and fourth prosthetic components while within the patient, where the tibia portion includes the third and the fourth prosthetic components connected to the fifth prosthetic component.

In general, in another aspect, the invention features a knee replacement prosthetic device including a first prosthetic component configured to be implanted into a patient while disconnected from a second prosthetic component, a second prosthetic component configured to be implanted into a patient while disconnected from the first prosthetic component, and one or more connectors configured to connect the first prosthetic component to the second prosthetic component after the first and the second prosthetic components are positioned within the patient, where the knee replacement prosthetic device includes a femur portion and a tibia portion and the femur portion is split along a coronal plane into the first and the second prosthetic components.

In general, in another aspect, the invention features a method for implanting a prosthetic device including making an incision in a patient, inserting a first prosthetic component into the patient through the incision, inserting a second prosthetic component into the patient through the incision and connecting the first prosthetic component to the second prosthetic component within the patient. The prosthetic device is sagittally split into the first prosthetic component and the second prosthetic component.

Implementations of the invention may feature one or more of the following. Connecting the first prosthetic component to the second prosthetic component can include inserting one or more connectors into the patient and connecting the first prosthetic component to the second prosthetic component with the one or more connectors. The method can further include inserting one or more additional prosthetic components into the patient and connecting the first, second and one or more additional prosthetic components within the patient. In this implementation, the prosthetic device includes the connected first, second and one or more additional prosthetic components. The incision can be smaller than an incision required to insert the prosthetic device into the patient with the first prosthetic component connected to the second prosthetic component.

In general, in another aspect, the invention features a method for implanting a knee replacement prosthetic device including making an incision in a patient, inserting a first prosthetic component into the patient through the incision, inserting a second prosthetic component into the patient through the incision and connecting the first prosthetic component to the second prosthetic component within the patient. The knee replacement device includes a femur portion and where the femur portion is sagittally split into the first prosthetic component and the second prosthetic component. The method further includes inserting a third prosthetic component into the patient through the incision, inserting a fourth prosthetic component into the patient through the incision, and connecting the third prosthetic component to the fourth prosthetic component within the patient. The knee replacement device further includes a tibia portion, where the tibia portion is sagittally split into the third prosthetic component and the fourth prosthetic component.

Implementations of the invention can include one or more of the following. A fifth prosthetic component can be inserted into the patient through the incision and the fifth prosthetic component can be connected to the third and fourth prosthetic components. In this implementation, the tibia portion includes the fifth prosthetic component connected to the third and fourth prosthetic components. The method can include connecting the femur portion of the knee replacement device to a femur of the patient and connecting the tibia portion of the knee replacement device to a tibia of the patient. Connecting the tibia portion to a tibia includes connecting the third and fourth prosthetic components to the tibia. The incision made in the patient can be smaller than an incision required to insert the femur portion with the first prosthetic component connected to the second prosthetic component into the patient. The incision made in the patient can be smaller than an incision required to insert the tibia portion with the third prosthetic component connected to the fourth prosthetic component into the patient.

Implementations of the invention can realize one or more of the following advantages. The size of an incision required to be made in a patient to implant a prosthetic device can be substantially smaller than is required to implant a conventional prosthetic device. The smaller an incision, the more quickly and fully a patient recovers from the implant surgery.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 shows a side view of a knee replacement prosthesis attached to a patient's femur and tibia.

FIG. 2 shows a perspective view of a knee replacement prosthesis.

FIG. 3A shows a perspective view of a tibia portion of a knee replacement prosthesis.

FIG. 3B shows an exploded view of the tibia portion of FIG. 3A.

FIG. 4 shows a front view of the tibia portion shown in FIG. 3A.

FIG. 5 shows a rear view of the tibia portion shown in FIG. 3A.

FIG. 6 shows a side view of the tibia portion shown in FIG. 3A.

FIG. 7 shows a top view of the tibia portion shown in FIG. 3A.

FIG. 8 shows a bottom view of the tibia portion shown in FIG. 3A.

FIG. 9A shows a perspective view of a femur portion of a knee replacement prosthesis.

FIG. 9B shows an exploded view of the femur portion of FIG. 9A.

FIG. 10 shows a front view of the femur portion shown in FIG. 9A.

FIG. 11 shows a rear view of the femur portion shown in FIG. 9A.

FIG. 12 shows a side view of the femur portion shown in FIG. 9A.

FIG. 13 shows mating edges of prosthetic components with a tongue and groove configuration.

FIG. 14 shows mating edges of prosthetic components with an angled configuration.

FIG. 15 is a flowchart showing a process for implanting a knee replacement prosthesis into a patient.

Like reference symbols in the various drawings indicate like elements.

### DETAILED DESCRIPTION

A prosthetic device is described including at least two prosthetic components configured to be inserted into a patient separately and connected to one another while within the patient, allowing for a minimal incision to be made in the patient to implant the prosthetic device.

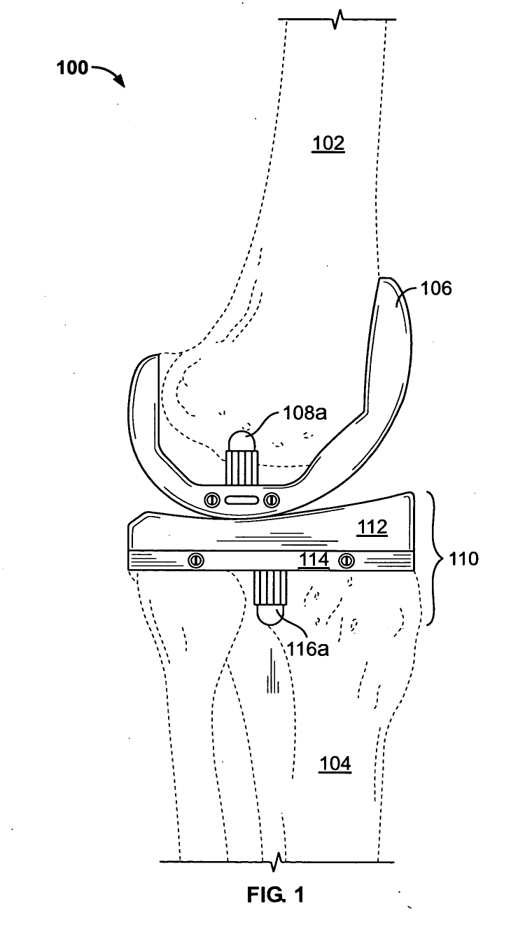

Referring to FIG. 1, a side view of a knee replacement prosthesis 100 implanted into a patient's femur 102 and tibia 104 is shown. The prosthesis includes a femur portion 106 attached to the femur 102 by way of two connectors 108a and 108b. The prosthesis further includes a tibia portion 110 attached to the tibia 104. The tibia portion 110 includes a cap 112 and base 114, where the base 114 connects to the tibia 104 by way of two connectors 116a and 116b. Other configurations and numbers of connectors 108 and 116 can be used, including a sagitally split connector.

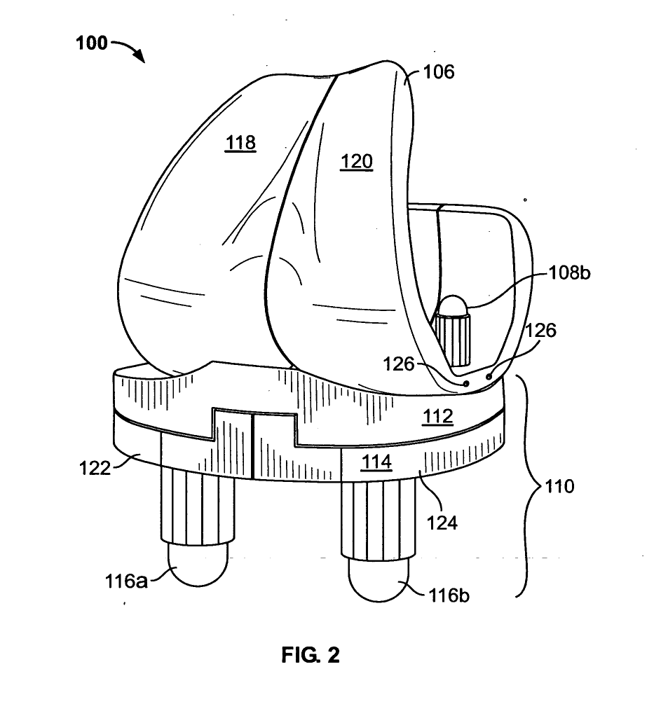

Referring to FIG. 2, a perspective view of the prosthesis 100 is shown. The femur portion 106 is sagittally split from front to back into two prosthetic components 118 and 120. The base 114 of the tibia portion 110 is also sagittally split from front to back into two prosthetic components 122 and 124. The two prosthetic components 118, 120 forming the femur portion 106 are implanted into the patient separately, and connected to one another in situ. For example, in the implementation shown, the two prosthetic components 118, 120 are connected to one another by two connectors 126, for example, screws, although other configurations of connectors can be used. Similarly, the two prosthetic components 122, 124 forming the base 114 of the tibia portion 110 are implanted into the patient separately and connected to one another in situ. In the implementation shown, the two prosthetic components 122, 124 are connected to one another by two connectors 128, e.g., screws, although other configurations of connectors can be used. Forming the femur and tibia portions 106, 110 with separate and connecting components allows the prosthesis 100 to be implanted into the patient using a significantly smaller incision then when inserting a conventional knee replacement prosthesis.

For clarity, the term sagittally split is used herein to refer to a split dividing a portion into left and right components if looking at a front view of the portion, as compared to a coronal split which refers to a split dividing a portion into front and rear components. The left and right components if sagittally split, or the front and rear components if a coronal split may or may not be substantially symmetric to one another.

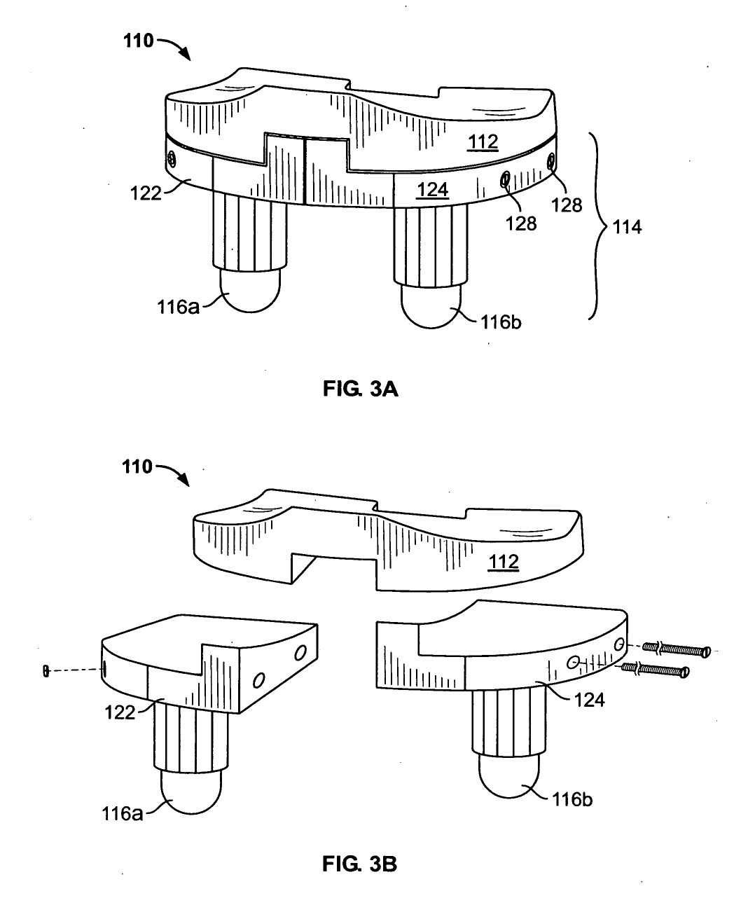

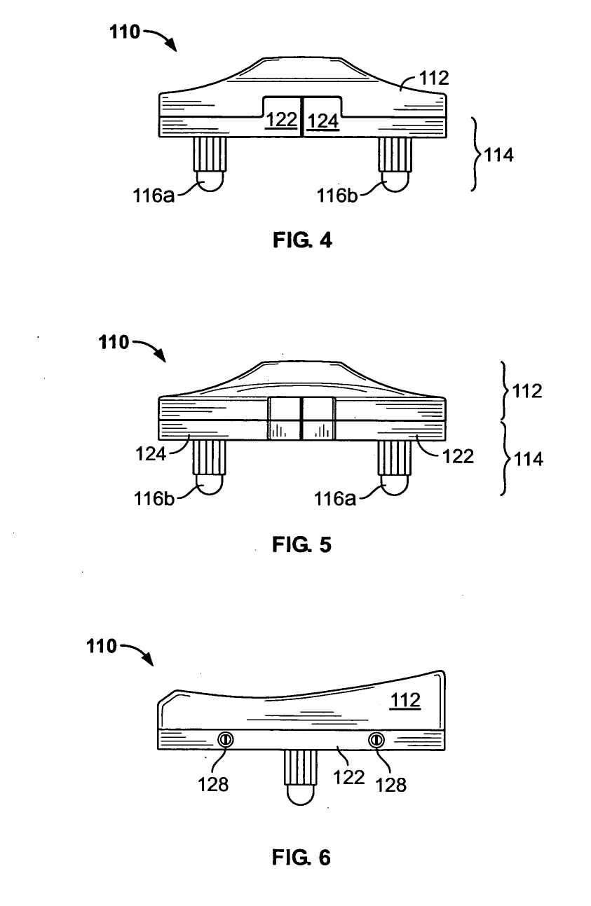

Referring to FIGS. 3-8, various views of the tibia portion 110 of the prosthesis 100 are shown. FIG. 3A shows a perspective view of the tibia portion 110 and FIG. 3B shows an exploded view of the tibia portion 110. The cap 112 is formed as one piece, and is implanted into the patient separately from the two prosthetic components 122, 124 forming the base 114 of the tibia portion 110. Alternatively, in another embodiment, the cap 112 can be formed from two or more pieces that are implanted separately into the patient and connected to one another in situ. FIGS. 4 and 5 show front and rear views of the tibia portion 110 respectively. The tibia portion 110 connects to the patient's tibia 104 by way of one or more connectors, such as the connectors 116a and 116b.

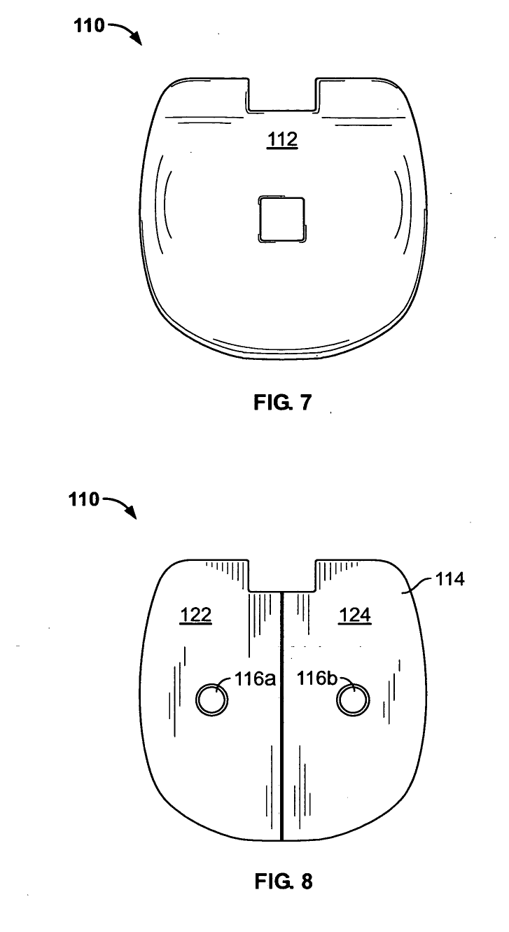

Referring to FIG. 6, a side view of the tibia portion 110 is shown. The two prosthetic components 122, 124 are connected to one another in situ by connectors 128 before or after the base 114 is connected to the patient's tibia 104. The base 114 can be connected to the tibia 104 using techniques for connecting a conventional knee prosthesis to a patient's tibia, or any other convenient technique. FIGS. 7 and 8 show top and bottom views of the tibia portion 110 respectively.

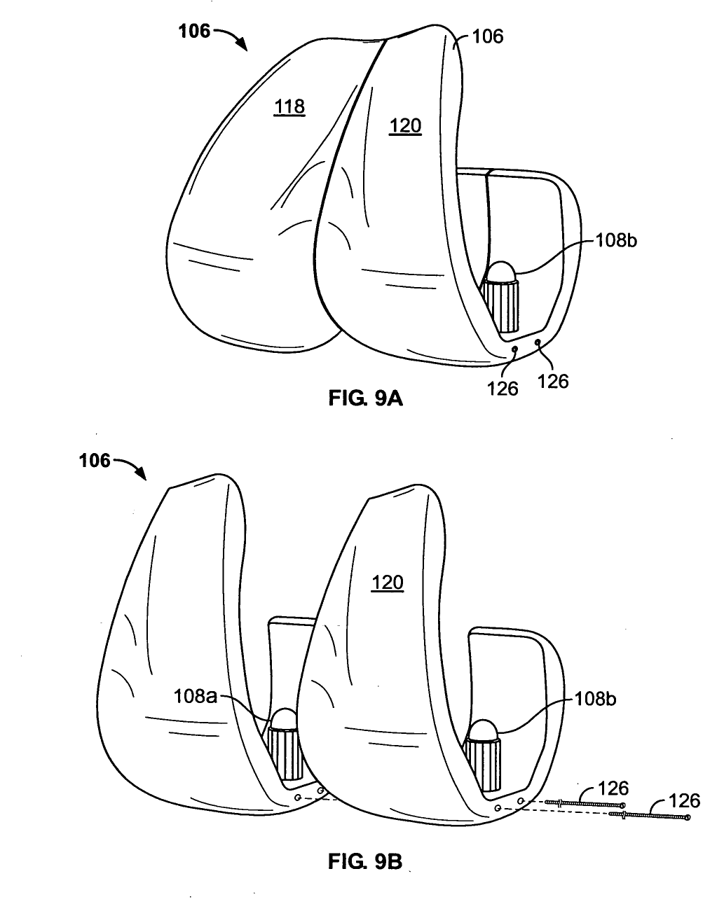

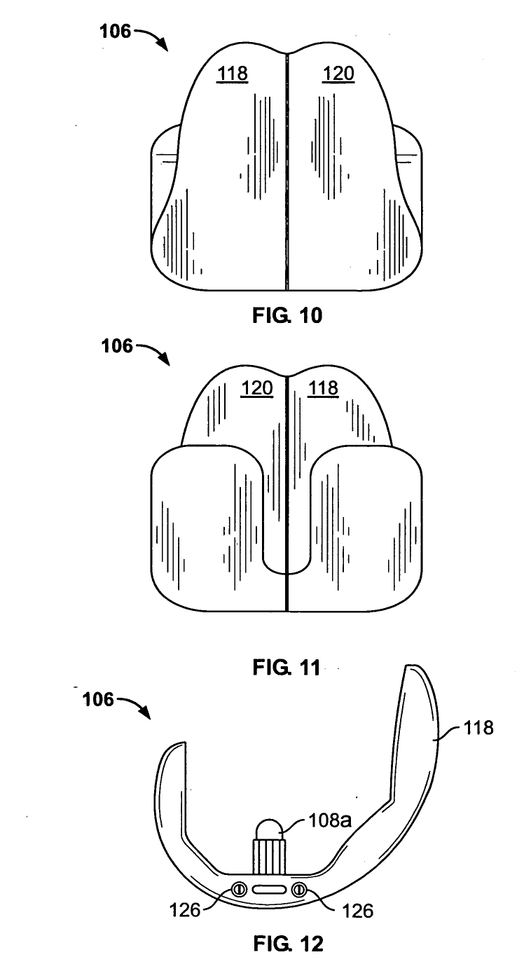

Referring to FIGS. 9-12, various views of the femur portion 106 of the prosthesis 100 are shown. FIG. 9A shows a perspective view of the femur portion 106, with the sagittal split between the two prosthetic portions 118 and 120 clearly shown. FIG. 9B shows an exploded view of the femur portion 106. FIG. 12 shows a side view of the femur portion 106. The connectors 126 holding the two prosthetic components 118 and 120 together are visible. More or fewer connectors 126 can be used, as well as a combination of different types of connectors. The femur portion 106 includes connectors 108a and 108b that attach into the patient's femur, as shown in FIG. 1. The femur portion 106 can be connected to the femur 102 using techniques for connecting a conventional knee prosthesis to a patient's femur, or any other convenient technique. FIGS. 10 and 11 show front and rear views of the femur portion 106 respectively.

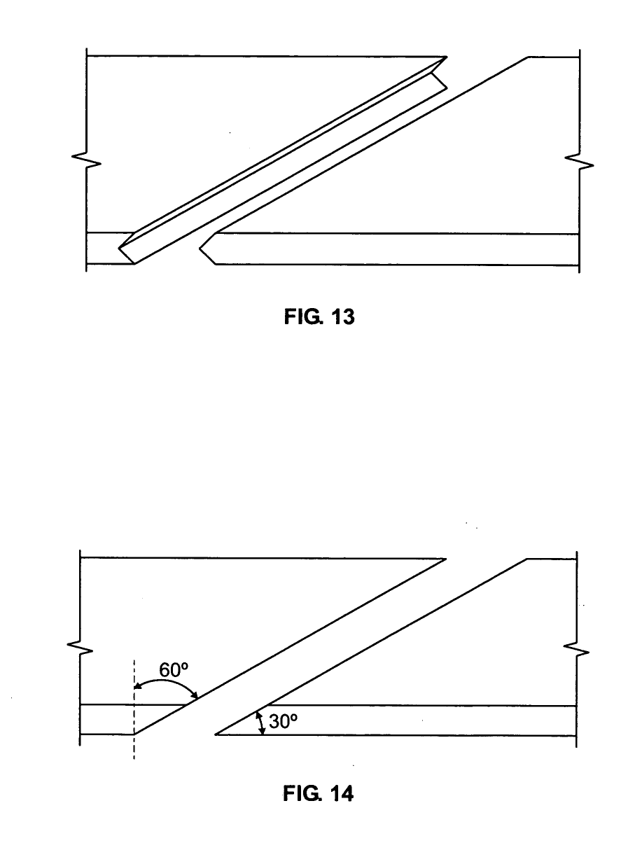

The two prosthetic components of either the femur portion 106 or tibia portion 110 include mating edges configured to provide a smooth, tight fit to one another. For example, in one implementation, the first and second prosthetic components 118 and 120 forming the femur portion 106 can be configured with tongue and groove edges respectively along the interface at the sagittal split. The first and second prosthetic components 118 and 120 therefore fit together with a tongue and groove connection (see FIG. 13). In another implementation, the mating edges can be configured with the edge of the first prosthetic component 118 formed at an angle of approximately 60° and the edge of the second prosthetic component 120 formed at an angle of approximately 30° (see FIG. 14). The first and second prosthetic components 118 and 120 can thereby mate to form a substantially smooth connection along the sagittal split. Other configurations of the first and second prosthetic components 118 and 120 can be used. Similarly, the edges of the third and fourth prosthetic components 122 and 124 forming the base 114 of the tibia portion 110 are configured to provide a smooth, tight fit to one another.

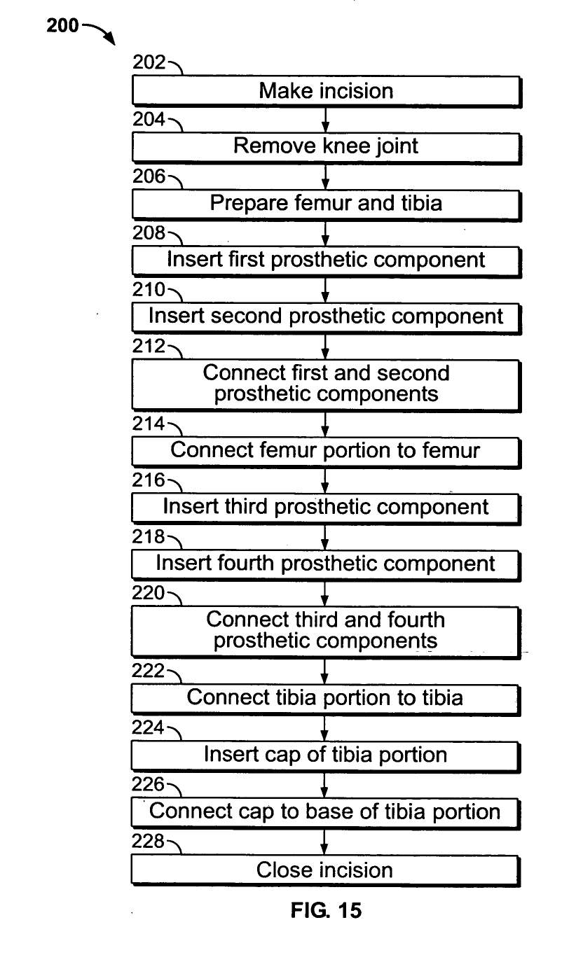

As mentioned above, forming the femur and tibia portions 106, 110 with separate and connecting components allows the prosthesis 100 to be implanted into the patient using a significantly smaller incision then when inserting a conventional knee replacement prosthesis. Referring to FIG. 15, a flowchart illustrating a process 200 for implanting the prosthesis 100 into the patient is shown. The prosthesis 100 is implanted under surgical conditions. For illustrative purposes, only actions taken to implant the prosthesis 100 are described, although it should be understood that other actions typical under surgical conditions may be undertaken.

An incision is made into the patient's skin and tissue in the region of the patient's knee (step 202). The patient's knee joint is removed (step 204). The patient's femur and tibia are prepared to receive the prosthesis (step 206), for example, holes are drilled into the ends of the femur 102 and tibia 104 configured to receive the connectors 108a, 108b and 116a, 116b of the femur portion 106 and tibia portion 110 respectively. The first and second prosthetic components 118 and 120 forming the femur portion 106 are inserted into the patient through the incision (steps 208 and 210). While in situ, the first and second prosthetic components 118 and 120 are connected to one another, e.g., using connectors 126 (step 212). The femur portion 106 is positioned into the patient's femur 102 and connected to the femur 102 (step 214).

The third and fourth prosthetic components 122 and 124 forming the base 114 of the tibia portion 110 are inserted into the patient through the incision (steps 216 and 218). While in situ, the third and fourth prosthetic components 122 and 124 are connected to one another, e.g., using connectors 128 (step 220). The tibia portion 110 is positioned into the patient's tibia 104 and connected to the tibia 104 (step 222). The cap 112 is inserted into the patient through the incision (step 224) and is connected to the upper surface of the base 114 (step 226). For example, in one implementation, the cap 112 is attached by a snap fit connection to the upper surface of the base 114. Once the prosthesis 100 is connected to the femur 102 and tibia 104, the incision can be surgically closed (step 228).

The prosthesis 100 shown above in FIGS. 1-12 is exemplary, and other configurations of a prosthesis 100 can be used, where the femur and tibia portions are segmented into two or more separate components that are connected together in situ. In the implementation described, the femur and tibia portions were sagittally split from front to back. Alternatively the femur and tibia portions can be split along different planes and in different directions, and the sagittal split shown above is just one possibility. For example, the femur and tibia portions can be split in a coronal direction providing front and back components.

The femur portion 106 and base 114 of the tibia portion 110 can be formed from any suitable material including, for example, stainless steel or cobalt chrome. The cap 112 of the tibia portion 110 can be formed from any suitable material including, for example, a high density polyethylene. The connectors 126 and 128 connecting the components of the femur and tibia portions together can be formed from any suitable material. Preferably, if the femur and tibia portions are formed from a metal, the connectors 126 and 128 are formed from the same metal.

The implementation of the prosthesis 100 described above is a knee replacement prosthesis including at least five components. However, it should be understood, that the prosthesis 100 can include more or fewer components. For example, the femur portion 106 can be formed from three components that are inserted into the patiently separately and connected together in situ. Other numbers of components can be used. Additionally, the prosthesis 100 can be a different type of implantable prosthesis, including finger, hip, shoulder, elbow, wrist, back, ankle and foot joints, to name a few. The prosthesis 100 is configured into two or more components that can be connected together in situ to minimize the shape and size of incision required to insert the components into the patient. For example, a conventional knee replacement surgery requires an incision of approximately 10 inches in length to implant a conventional knee replacement prosthesis, although some more recent techniques can do an implantation with an incision of approximately 4 inches. The prosthesis 100 for knee replacement described above can be implanted into a patient with an incision of approximately 3 inches or less. Accordingly, the length of the incision can be reduced by 25% or more, a significant reduction.

A number of embodiments of the invention have been described. Nevertheless, it will be understood that various modifications may be made without departing from the spirit and scope of the invention. The logic flow depicted in FIG. 15 does not require the particular order shown, or sequential order, to achieve desirous results, and the steps of the invention can be performed in a different order. Accordingly, other embodiments are within the scope of the following claims.

What is claimed is:

1. A prosthetic device, comprising:
a first prosthetic component configured to be implanted into a patient while disconnected from a second prosthetic component;
a second prosthetic component configured to be implanted into a patient while disconnected from the first prosthetic component; and
one or more connectors configured to connect the first prosthetic component to the second prosthetic component after the first and the second prosthetic components are positioned within the patient, where the prosthetic device is sagittally split into the first and the second prosthetic components.

2. The prosthetic device of claim 1, where:
the prosthetic device comprises a knee replacement device including a femur portion; and
the femur portion comprises the first prosthetic component connected to the second prosthetic component, the femur portion configured to connect to the patient's femur.

3. The prosthetic device of claim 2, the prosthetic device further comprising a tibia portion configured to connect to the patient's tibia.

4. The prosthetic device of claim 3, where the tibia portion includes a base, the base comprising:
a third prosthetic component configured to be implanted into the patient while disconnected from a fourth prosthetic component;
a fourth prosthetic component configured to be implanted into the patient while disconnected from the third prosthetic component;
one or more second connectors configured to connect the third prosthetic component to the fourth prosthetic component after the third and the fourth prosthetic components are positioned within the patient, where the base is sagittally split into the third and the fourth prosthetic components.

5. The prosthetic device of claim 4, where the tibia portion further comprises a cap, the cap comprising:
a fifth prosthetic component configured to be implanted into the patient, and configured to connect to the third and fourth prosthetic components while within the patient.

6. The prosthetic device of claim 4, where the one or more second connectors comprise screws.

7. The prosthetic device of claim 1, where the one or more connectors comprise screws.

8. A knee replacement device, comprising:
a femur portion sagittally split into a first prosthetic component and a second prosthetic component, the femur portion including:
the first prosthetic component configured to be implanted into a patient while disconnected from a second prosthetic component;
the second prosthetic component configured to be implanted into a patient while disconnected from the first prosthetic component; and
one or more first connectors configured to connect the first prosthetic component to the second prosthetic component after the first and the second prosthetic components are positioned within the patient; and
a tibia portion sagittally split into a third prosthetic component and a fourth prosthetic component, the tibia portion including:
the third prosthetic component configured to be implanted into a patient while disconnected from a fourth prosthetic component;
the fourth prosthetic component configured to be implanted into a patient while disconnected from the third prosthetic component; and
one or more second connectors configured to connect the third prosthetic component to the fourth prosthetic component after the third and the fourth prosthetic components are positioned within the patient.

9. The knee replacement device of claim 8, further comprising:
a fifth prosthetic component configured to be implanted into the patient, and configured to connect to the third and fourth prosthetic components while within the patient, where the tibia portion comprises the third and the fourth prosthetic components connected to the fifth prosthetic component.

10. A knee replacement prosthetic device, comprising:
a tibia portion; and
a femur portion, where the femur portion is split along a coronal plane into a first prosthetic component and a second prosthetic components, the femur portion including:
the first prosthetic component configured to be implanted into a patient while disconnected from a second prosthetic component;
the second prosthetic component configured to be implanted into a patient while disconnected from the first prosthetic component; and
one or more connectors configured to connect the first prosthetic component to the second prosthetic component after the first and the second prosthetic components are positioned within the patient.

11. A method for implanting a prosthetic device, comprising:
making an incision in a patient;
inserting a first prosthetic component into the patient through the incision;
inserting a second prosthetic component into the patient through the incision;
connecting the first prosthetic component to the second prosthetic component within the patient, where the prosthetic device is sagittally split into the first prosthetic component and the second prosthetic component.

12. The method of claim 11, where connecting the first prosthetic component to the second prosthetic component comprises inserting one or more connectors into the patient and connecting the first prosthetic component to the second prosthetic component with the one or more connectors.

13. The method of claim 11, further comprising:
inserting one or more additional prosthetic components into the patient; and
connecting the first, second and one or more additional prosthetic components within the patient, the prosthetic device comprising the connected first, second and one or more additional prosthetic components.

14. The method of claim 11, where the incision is smaller than an incision required to insert the prosthetic device into the patient with the first prosthetic component connected to the second prosthetic component.

15. A method for implanting a knee replacement prosthetic device, comprising:
making an incision in a patient;
inserting a first prosthetic component into the patient through the incision;
inserting a second prosthetic component into the patient through the incision;
connecting the first prosthetic component to the second prosthetic component within the patient, where the knee replacement device includes a femur portion and the femur portion is sagittally split into the first prosthetic component and the second prosthetic component;
inserting a third prosthetic component into the patient through the incision;
inserting a fourth prosthetic component into the patient through the incision; and
connecting the third prosthetic component to the fourth prosthetic component within the patient, where the knee replacement device further includes a tibia portion and the tibia portion is sagittally split into the third prosthetic component and the fourth prosthetic component.

16. The method of claim 15, further comprising:
inserting a fifth prosthetic component into the patient through the incision; and
connecting the fifth prosthetic component to the third and fourth prosthetic components, where the tibia portion further comprises the fifth prosthetic component connected to the third and fourth prosthetic components.

17. The method of claim 15, further comprising:
connecting the femur portion of the knee replacement device to a femur of the patient; and
connecting the tibia portion of the knee replacement device to a tibia of the patient.

18. The method of claim 17, where connecting the tibia portion to a tibia comprises connecting the third and fourth prosthetic components to the tibia.

19. The method of claim 15, where the incision is smaller than an incision required to insert the femur portion with the first prosthetic component connected to the second prosthetic component into the patient.

20. The method of claim 15, where the incision is smaller than an incision required to insert the tibia portion with the third prosthetic component connected to the fourth prosthetic component into the patient.
